(19) United States
(12) Patent Application Publication
(10) Pub. No.: US 2013/0295669 A1
(43) Pub. Date: Nov. 7, 2013

(54) METHOD FOR CONTROLLING BINDING OF CELLS TO A SUBSTRATE

(71) Applicants: Judith Schenk, Stuttgart (DE); Christian Van Den Bos, Greven (DE); Claudia Rosenbaum, Duesseldorf (DE); Ying Nie, Frederick, MD (US)

(72) Inventors: Judith Schenk, Stuttgart (DE); Christian Van Den Bos, Greven (DE); Claudia Rosenbaum, Duesseldorf (DE); Ying Nie, Frederick, MD (US)

(73) Assignee: LONZA COLOGNE GMBH, Koeln (DE)

(21) Appl. No.: 13/884,302

(22) PCT Filed: Nov. 9, 2011
(86) PCT No.: PCT/EP2011/069773

§ 371 Date: Jul. 18, 2013

(30) Foreign Application Priority Data
Nov. 9, 2010 (EP) 10014403.9

(51) Int. Cl. C12N 5/0775

(52) U.S. Cl. CPC C12N 5/0675
USPC 435/375; 435/402

(57) ABSTRACT

The invention relates to a method for promoting the adhesion of cells to a substrate to which these cells usually have no or only low affinity, wherein the adhesion of the cells to the substrate is promoted by supplying the cells with the non-muscle myosin II inhibitor Blebbistatin so as to enable the cells to attach to surfaces to which they otherwise would not have sufficient affinity. Surprisingly, supplying the cells with the inhibitor enhances the capability of these cells to attach to surfaces to which they usually have no or only low affinity, for example, PTFE (Teflon®). The invention further concerns uses of the non-muscle myosin II inhibitor Blebbistatin and devices having at least one surface which is coated with cells that have no or only low affinity to said surface.

### BACKGROUND OF THE INVENTION

The invention relates to a method for promoting the adhesion of cells to a substrate to which these cells usually have no or only low affinity. The invention further concerns uses of the non-muscle myosin II inhibitor Blebbistatin and devices having at least one surface which is coated with cells that have no or only low affinity to said surface.

### PRIOR ART

Most of the currently used therapeutic cells (including adult stem cells) are propagated in anchorage/attachment-dependent fashion and this is also true for potential alternatives such as embryonic stem cells and induced pluripotent stem cells (iPS). However, such propagation is inherently associated with severe limitations in scale and economy. For cell culture expansion, cells are typically seeded onto planar surfaces to which they attach within a matter of hours, left to proliferate and harvested by means of proteolytic enzymes and/or calcium removal. Attachment is not a homogeneous process but rather occurs through particular zones, so-called focal adhesion zones. Once attached, cells exert tension by means of internal motor proteins. Thus, the cytoskeleton becomes organized and functions in a variety of processes including transport, scaffolding and cell survival. Upon de-attaching cells, these quickly constrict as a function of the internal tension build up by motor proteins and the previously organized internal structures (cytoskeletal and other) are disturbed. If not allowed to re-attach to surfaces, such cells will initiate cell death programs which have been described as attachment-dependent apopotosis/anoikis.

US-A-2010 0009 442 teaches that inhibitors (such as Y-27632, H-1152 or Fasudil) of Rho-associated coiled kinase (ROCK), which is an effector molecule of Rho GTPase and known to control vascular constriction and nerve axon extension, can be used as inhibitors of apoptosis/anoikis and thus improve the survival and/or proliferation rate of pluripotent stem cells, especially embryonic stem cells. In the presence of ROCK inhibitors, the stem cells can be cultured without feeder cells and/or serum prior and/or after subcloning or passaging.

The major cytoskeletal motor protein responsible for generating cell tension is non-muscle myosin II (referred to as myosin II). In their attempt to dissect cytokinesis, Straight et al. (2003) identified a particular small molecule inhibitor of non-muscle myosin II. This highly active small molecule inhibits cell blebbing and was termed Blebbistatin. This compound is cell-permeant, benign and, importantly, readily reversible.

Walker et al. (2010) disclose the use of Blebbistatin for enhancing the survival of human pluripotent stem cells, including human embryonic stem cells and induced pluripotent stem cells. Treatment with Blebbistatin increases the survival rate of human pluripotent stem cells under clonal density and suspension conditions. Moreover, in combination with a synthetic matrix, Blebbistatin supports a defined environment for self-renewal of the stem cells.

US-A-2010 0216 181 teaches cultivation of pluripotent stem cells in a medium that is free of serum and feeder cells using Blebbistatin or ROCK inhibitors as survival factor. Here, large numbers of cells are generated by cultivating the cells in spinner flasks or bioreactors.

Attachment-dependent cells can be produced in large quantities in bioreactors on microcarriers (e.g. beads), which will increase the surface area on which the cells can be grown, while culturing the cells under suspension conditions. For example, a method for large-scale production of stem cells, including pluripotent and embryonic stem cells, is known from US-A-2010 0093 083. Herein, the cells are cultivated in serum-free medium containing a plurality of microcarriers. The cells are allowed to adhere to the microcarriers and expanded in a bioreactor under controlled conditions. The medium may be supplemented with additional components that promote proliferation and survival of the stem cells or prevent differentiation, for example inhibitors of the enzymes GSK3 or MEK. After expansion, the cells are separated from the microcarriers using an enzymatic or non-enzymatic cell dissociation reagent. However, it is a drawback of this method that only microcarriers can be used to which the cells have sufficient affinity.

### SUMMARY OF THE INVENTION

It is an object of the invention to provide a method for promoting the adhesion of cells to a substrate to which these cells usually have no or only low affinity.

This object is solved by a method wherein the adhesion of the cells to the substrate is promoted by supplying the cells with the non-muscle myosin II inhibitor Blebbistatin so as to enable the cells to attach to surfaces to which they otherwise would not have sufficient affinity. Surprisingly, supplying the cells with the inhibitor enhances the capability of these cells to attach to surfaces to which they usually have no or only low affinity. Accordingly, Blebbistatin can be used in an advantageous manner as a kind of enhancer which enables the user to promote attachment of cells to specific substrates, such as microcarriers for suspension culture or surfaces coated with, e.g., Teflon®. Thus, Blebbistatin is a useful tool to broaden the choice of suitable substrates for cell cultures so that cell culture methods relying on attachment of cells to microcarriers can be optimized and thus significantly improved. Improving and optimizing of culture conditions is facilitated since there are much more options in respect of the substrate to which the cells to be cultured can be adhered. Moreover, colonizing surfaces that are optimized for other needs but usually not a proper substrate for living cells, for example, surfaces of some medical devices, can be achieved by adding Blebbistatin to the cells. Thus, treating the cells with the inhibitor results in the capability of these cells to attach to and proliferate on surfaces that would otherwise not be suitable for culturing these cells. By the method according to the invention the choice of substrates, e.g. microcarriers or medical devices, suitable for culturing and/or expanding cells is significantly broadened.

Blebbistatin is a small molecule which, when applied to cell cultures, minimizes cell death induced by lack of attachment and promotes single cell cultures. In contrast to ROCK inhibitors, Blebbistatin acts as a direct, non-competitive inhibitor of non-muscle myosin II. It does not interfere with a complex signaling cascade but rather directly targets non-muscle myosin II and its interaction with actin by binding to the myosin-ADP-Pi complex. Importantly, Blebbistatin is an easily adjustable and reversible (benign) inhibitor of myosin II. Blebbistatin enhances the capability of cells, especially attachment-dependent cells, to attach to surfaces to which they otherwise would not have sufficient affinity. Accordingly, Blebbistatin can be used as an adhesion trigger which renders controlling of attachment of cells to specific substrates possible. Moreover, Blebbistatin greatly enhances suspension survival of cells otherwise strictly dependent on attachment. The substance can be added readily to media when required.

In a preferred embodiment of the invention the substrate is a non-stick material or at least in part coated with a non-stick material. The non-stick material may comprise polytetrafluoroethylene (PTFE, trade name (DuPont): Teflon®), polyfluoroethylene (PFE), perfluoroalkoxy (PFA), fluorinated ethylene propylene (FEP), a titanium dioxide compound or a titanium dioxide comprising composition, or any combination thereof.

Preferably, the substrate is a surface of a medical device, preferably a stent, a patch or an artificial blood vessel or organ. As such devices are often made of or coated with a material that is usually not suitable for cell culture, for example, to avoid undesired attachment of blood cells, proteins, or the like, it is usually difficult to colonize those devices with cells. However, it is sometimes desirable to coat such devices with living cells, preferably stem cells, so that they can be used as implants, replacement tissue, repair probes, or the like. Also, rejection of implants by the immune system may be efficiently suppressed by coating the implants with autologous cells. Advantageously, according to the invention, cells can be enabled to colonize medical devices by supplying them with Blebbistatin so as to prepare and improve these devices for several medical applications.

In another preferred embodiment of the invention the substrate is a plurality of microcarriers so that the cells attached to said substrate can be cultivated under suspension conditions. In this embodiment, the surface area on which the cells can be grown is increased so that the cells can be produced in large quantities on microcarriers, preferably suitable beads, while culturing the cells under suspension conditions, preferably in a suitable bioreactor. According to the invention, commercially available microcarriers, such as Cytodex™ (GE Healthcare, GB), Hillex (SoloHill, USA) or the like, may be used, including but not limited to temperature-controlled microcarriers from which cells are removed by a change of temperature, preferably by lowering the temperature. For cell expansion, additional microcarriers may be added to the existing culture so as to provide additional attachment area for the increasing cell number. Thus, expansion of cell cultures is easily achieved by mere addition of more microcarriers. For certain applications it may be beneficial to use differently sized microcarriers.

In another preferred embodiment of the invention, the cells are attachment-dependent cells, in particular adult stem cells, preferably mesenchymal stem cells. However, the method according to the invention comprises all cell types whose affinity to a substrate can be enhanced by Blebbistatin.

As Blebbistatin enhances cell survival and decreases cell aggregation in suspension culture, the cells can be easily expanded under suspension culture conditions, preferably immobilized on microcarriers as outlined above.

For some applications it might be advantageous if the cells are suspended in a serum-reduced or serum-free culture medium.

In a particularly preferred embodiment of the invention, the cells are frozen and/or thawed in the presence of Blebbistatin and then brought in contact with said substrate. Advantageously, Blebbistatin stabilizes the cells in this case and hence has a protective effect on the cells while freezing and/or thawing them. That is, Blebbistatin increases the cryopreservation recovery of cells and adding Blebbistatin before freezing is beneficial for cryopreserving cells.

Accordingly, it is an important aspect of the invention to use Blebbistatin for promoting the adhesion of cells to a substrate to which they otherwise would not have sufficient affinity.

It is another important aspect of the invention to use Blebbistatin for coating a surface of a medical device, preferably a stent, a patch or an artificial blood vessel or organ, with cells that otherwise would have no or only low affinity to this surface.

A further important aspect of the invention concerns a device having at least one surface which is coated with cells that usually have no or only low affinity to this surface. The surface may comprise a non-stick material, preferably polytetrafluoroethylene (PTFE, trade name (DuPont): Teflon®), polyfluoroethylene (PFE), perfluoroalkoxy (PFA), fluorinated ethylene propylene (FEP), a titanium dioxide compound or a titanium dioxide comprising composition, or any combination thereof. In a preferred embodiment of the invention, the surface of the device is coated with attachment-dependent cells, in particular adult stem cells, preferably mesenchymal stem cells. In another preferred embodiment of the invention, the device is a medical device, preferably a stent, a patch or an artificial blood vessel or organ.

Basically, the concept according to the invention relates to the beneficial use of Blebbistatin for promoting the adhesion of cells to substrates to which they otherwise would not have sufficient affinity, preventing/reducing cell death during dislocation of cells into suspension culture, improving cell survival, permitting cell relocation to surfaces otherwise not suitable and finally potentiating cell proliferation of cells, preferably attachment dependent cells. The scope of cell types to benefit from the use of Blebbistatin in culture is very broad and includes the known therapeutically useful cells as well as candidates currently in development such as human embryonic stem cells (HES) and induced pluripotent stem cells (iPS). HES cells usually grow as aggregates and require frequent enzymatic segregation to allow for expansion. This process, however, suffers from cell death/loss and may benefit substantially from use of Blebbistatin and, indeed, use of Blebbistatin may be a step towards full suspension culture expansion of HES cells. While planar culture of adult stem cells and iPS cells is more robust, use of Blebbistatin is also beneficial during passaging and, importantly, as a step towards full suspension culture expansion of adult cells. While expansion of pluripotent adult stem cells is of particular interest, cell types for this concept shall not be limited to stem cells but rather include all attachment dependent cell types.

As used herein, an “inhibitor of non-muscle myosin II” or a “myosin II inhibitor” is a molecule or a plurality of molecules that inhibit(s) the function of non-muscle myosin II by targeting myosin II directly. Accordingly, the inventive concept comprises inhibitors that directly affect non-muscle myosin II. Blebbistatin, or any analogue or derivative thereof, is one example for such inhibitors. However, the invention is not limited to this example but may comprise other inhibitors having similar effects on non-muscle myosin II.

As used herein, an “attachment-dependent cell” is a cell that is generally not viable if suspended in a fluid but, in order to survive and grow, has to adhere to a solid substrate.

As used herein, an “adult stem cell”, also known as “somatic stem cell” is a multipotent cell that is capable of self-renewal and able to generate progeny of many different cell types, especially all cell types of the tissue or organ from which it originates. “Adult stem cells” include, but are not limited to, mesenchymal stem cells, hematopoietic stem cells, endothelial stem cells, neural stem cells and modifications thereof. “Adult stem cells” as used herein do not include induced pluripotent stem cells (iPS cells).

As used herein, an “induced pluripotent stem cell” (iPS cell) is a pluripotent cell derived from a non-pluripotent somatic cell that was reprogrammed by inducing expression of specific transcription factors. As being pluripotent, iPS cells are similar to embryonic stem cells.

As used herein, an “embryonic stem cell” is a pluripotent cell that is capable of differentiating into all three germ layers (endoderm, ectoderm and mesoderm) and germline cells.

The invention is further exemplarily described in detail with reference to the figures.

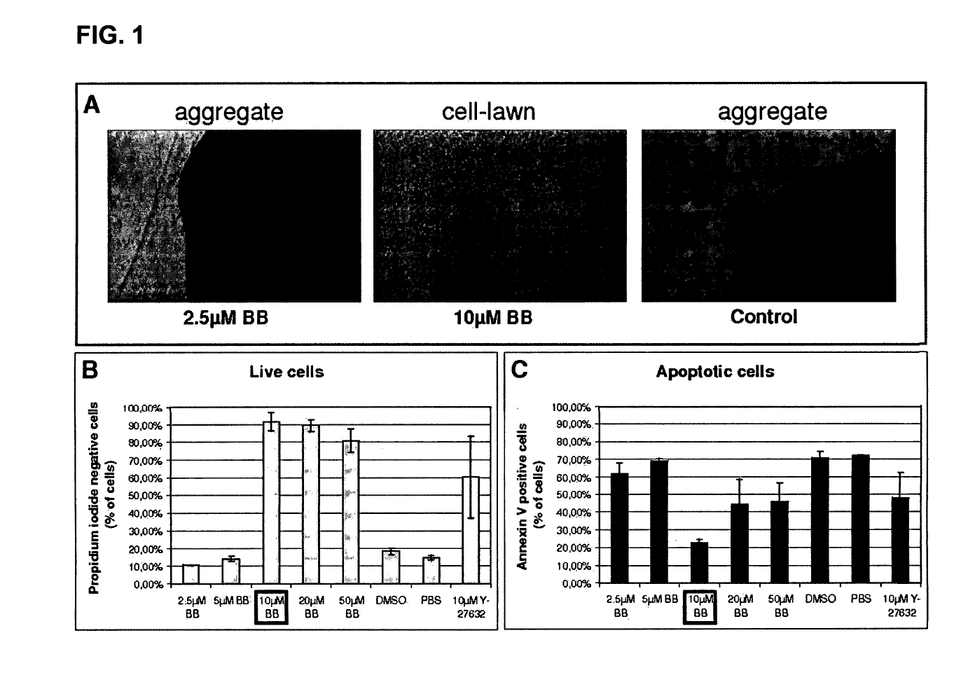

FIG. 1 shows the effect of Blebbistatin on cell aggregation (A: micrographs), cell survival (B: bar diagram) and apoptosis (C: bar diagram) of mesenchymal stem cells from bone marrow (BM-MSCs). Cell aggregation (A), cell survival (B) and apoptosis (C) were assayed in presence or absence of different Blebbistatin concentrations up to seven days. ROCK inhibitor (Y-27532) was used as a positive control. Cells were added to deep-wells not supporting adherent growth. Different concentrations of Blebbistatin and Y-27632 were tested in suspension (deep-well, 130 RPM) and adhesion (six-well) culture on BM-MSCs (media containing 10% FBS). It becomes apparent that Blebbistatin prevents or at least greatly reduces cell aggregation, enables cell survival and decreases apoptosis in deep well culture of mesenchymal stem cells.

A: Treatment with 10 μM Blebbistatin prevents cell clustering, whereas control cells (DMSO, PBS) and cells treated with less than 10 μM Blebbistatin formed a single aggregate.
B: Live cells were quantified by FACS using dye-exclusion assay (propidium iodide). In contrast to control cultures (DMSO, PBS) cells treated with Blebbistatin above 5 μM exhibited a degree of viability similar to cells in planar culture (˜90%). The survival-enhancing effect of Blebbistatin appeared concentration dependent peaking at 10 μM and decreasing at both higher and lower concentrations of Blebbistatin.
C: Apoptotic cells were quantified by FACS using Annexin V-assay (propidium iodide). In contrast to control cultures (DMSO, PBS) cells treated with 10 μM Blebbistatin were protected against apoptosis.

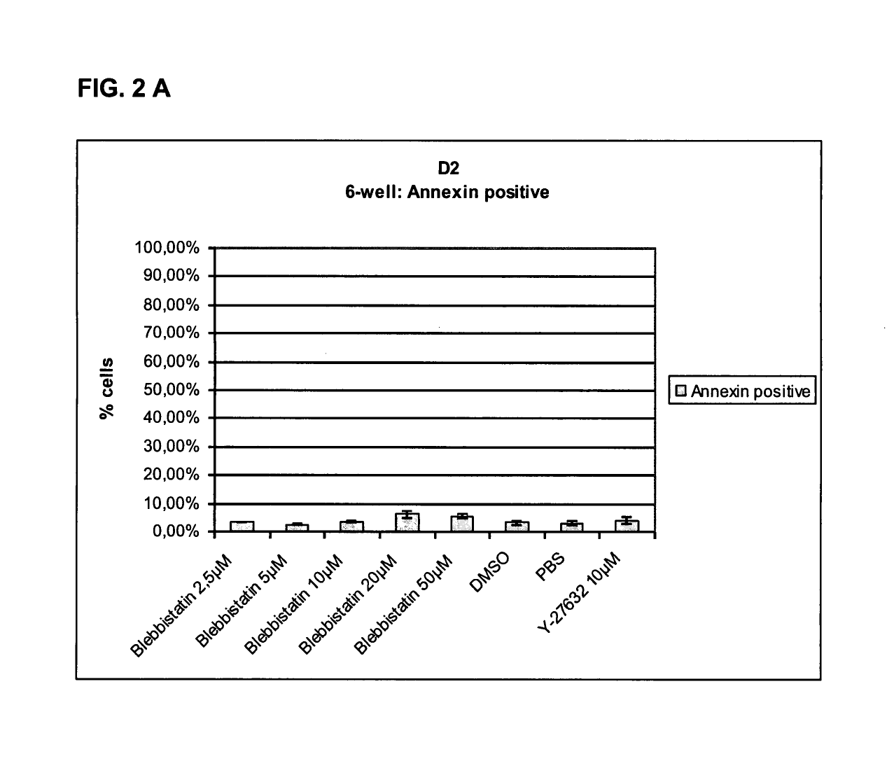

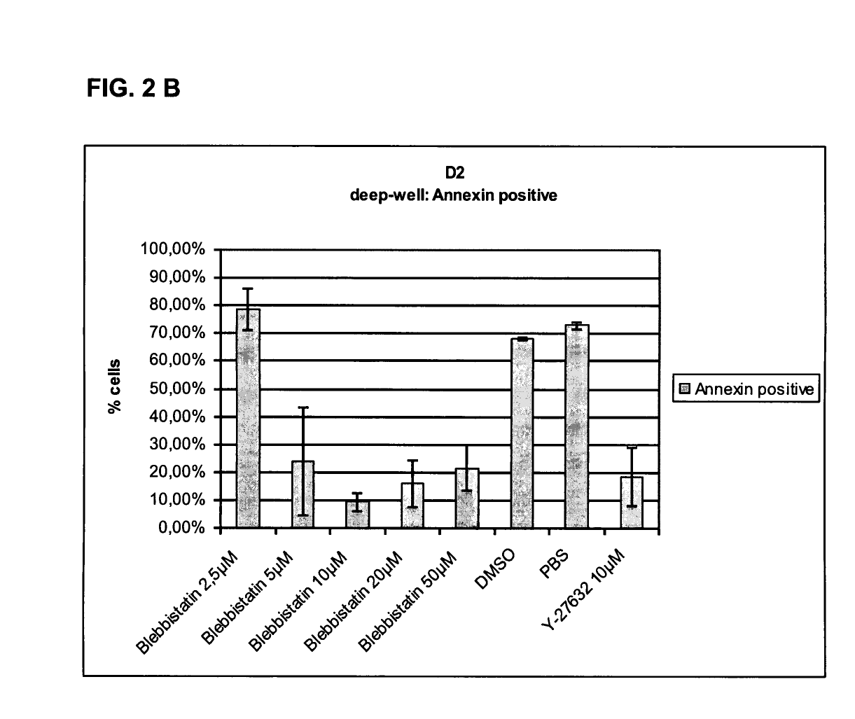

FIG. 2 shows apoptosis levels as revealed by AnnexinV staining and subsequent FACS-analysis of BM-MSCs treated with various inhibitor concentrations and cultured under adhesive/static conditions (A) or suspension/shaking conditions (B). Here, it becomes apparent that Blebbistatin eliminates cell death in suspension culture and shows an anti-apoptotic effect which is comparable or stronger than that of Y-27632. Below 5 μM Blebbistatin has merely a limited effect, wherein the greatest effect is achieved at 10 μM.

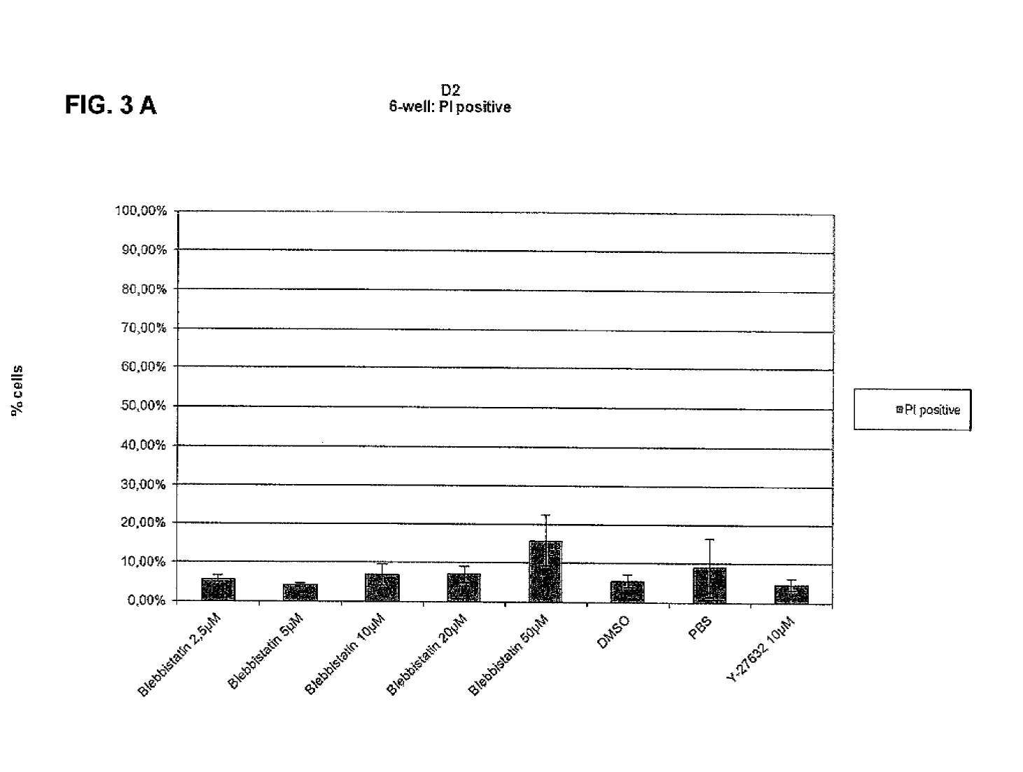

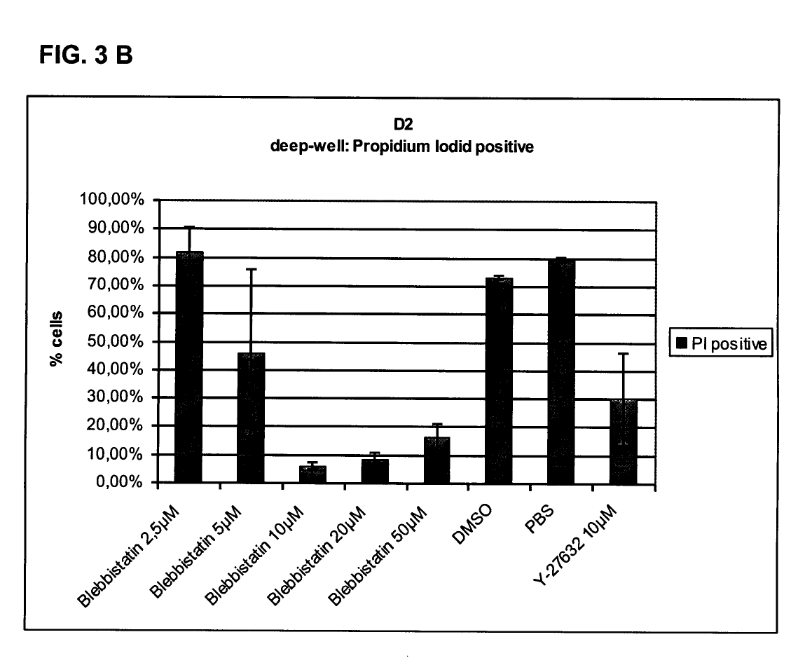

FIG. 3 shows cell death rates as revealed by propidium iodide and subsequent FACS-analysis of BM-MSCs treated with various inhibitor concentrations and cultured under adhesive/static conditions (A) or suspension/shaking conditions (B). This analysis shows that Blebbistatin significantly eliminates cell death, especially at concentrations of 10-50 μM, with an optimum at 10 μM.

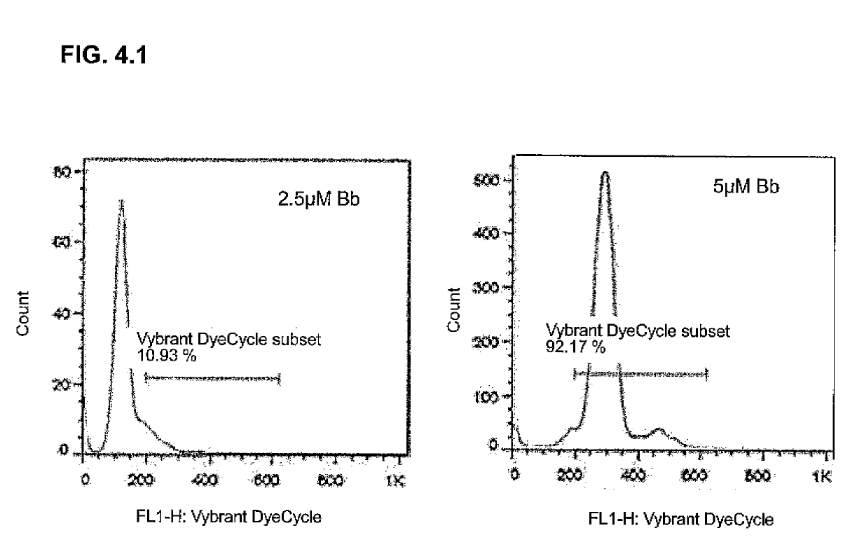

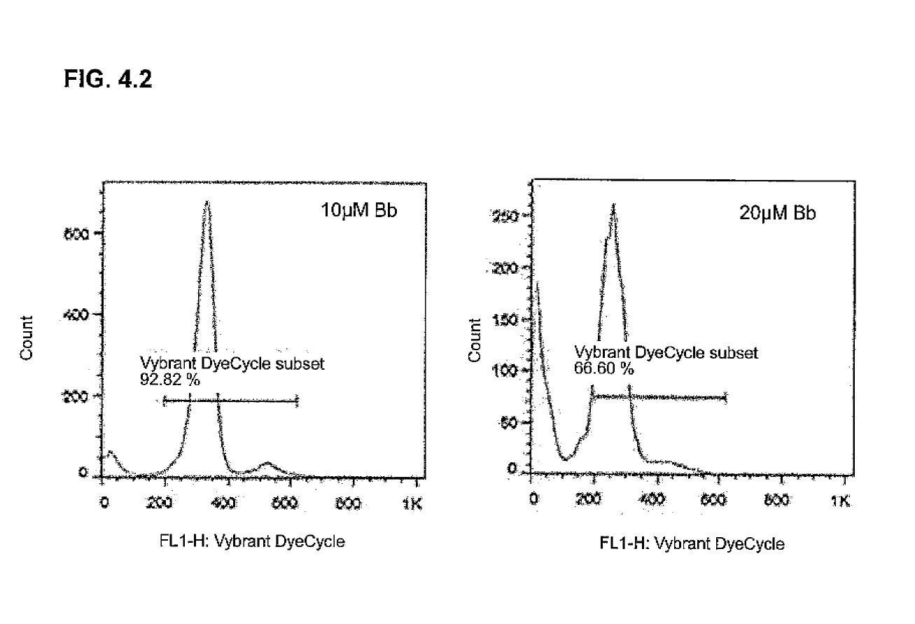

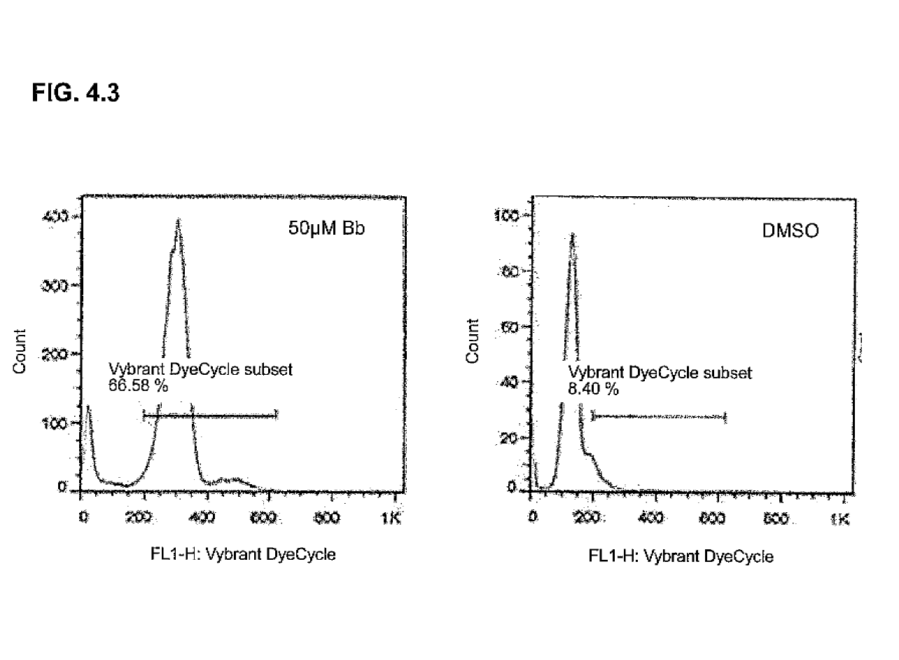

FIGS. 4.1-4.3 show cell cycle profiles (G1/G0 and G2) as revealed by propidium concentrations and cultured under suspension/shaking conditions in deep- wells. BM-MSC cultures were allowed to incorporate BrdU continuously and were sampled at different points of time (up to 3 days). Most cells are in G1/G0 phase (very dense culture), but there are also some G2-cells present. Absence of >4N cells (no multinucleated cells detected). As a result, there is no significant effect of Blebbistatin on cell cycle in suspension culture.

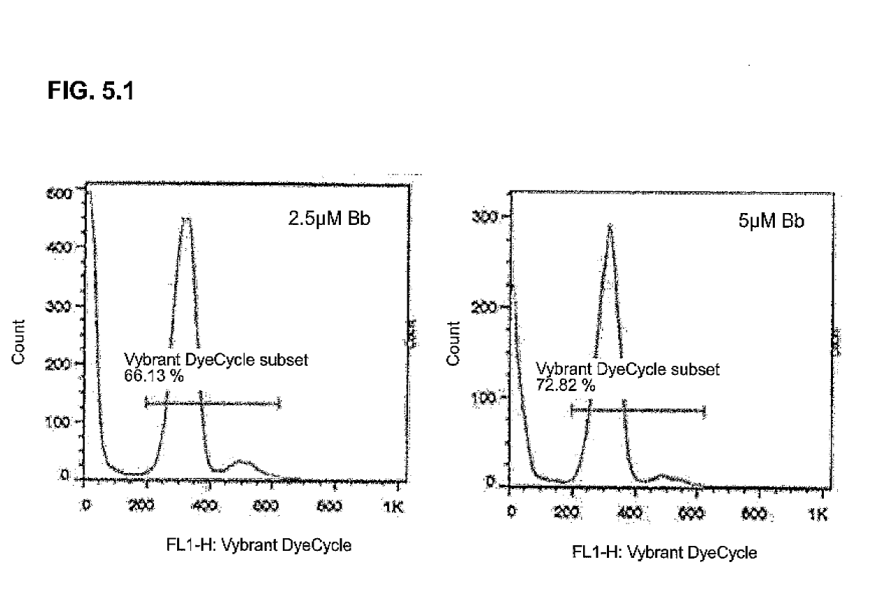

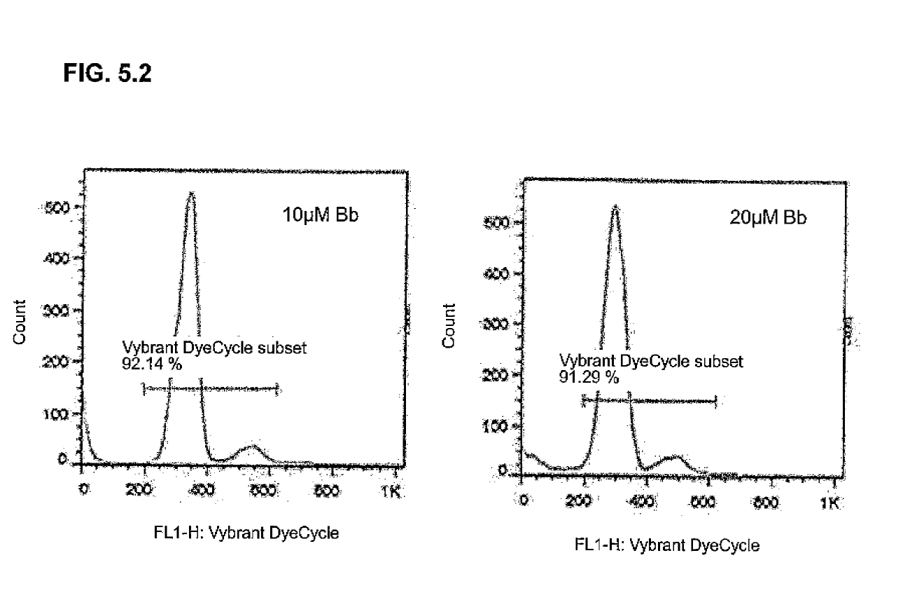

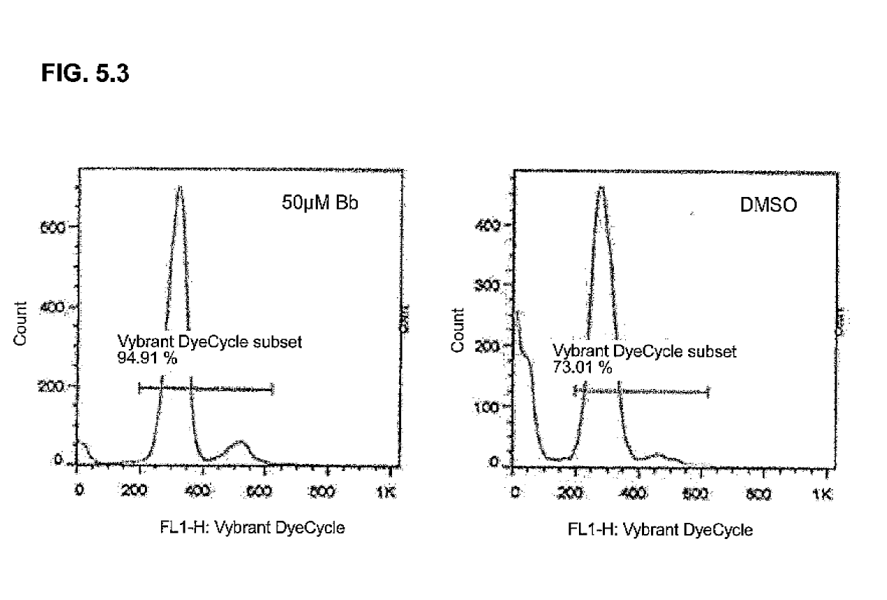

FIGS. 5.1-5.3 show cell cycle profiles (G1/G0 and G2) as revealed by propidium iodide and subsequent FACS-analysis of cells treated with various inhibitor concentrations and cultured under static conditions in six-wells. BM-MSC cultures were allowed to incorporate BrdU continuously and were sampled at different points of time (up to 3 days). Most cells are in G1/G0 phase (very dense culture), but there are also G2-cells present. Absence of >4N cells (no multinucleated cells detected). During continuous treatment in adherent T-flasks culture (G2/M) slight slowing down of the cell cycle was observed.

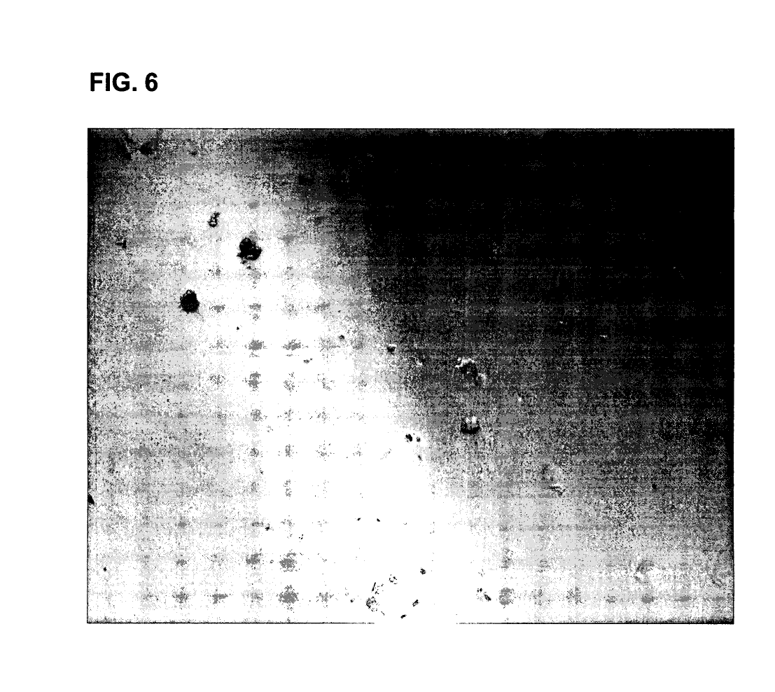

FIG. 6 shows BM-MSCs cultured on the hydrophobic side of a Lumox-biofoil bag without Blebbistatin treatment (control). Colony and attached cells observed after 48 h treatment with <0.1% DMSO are shown. No significant attachment of cells could be observed.

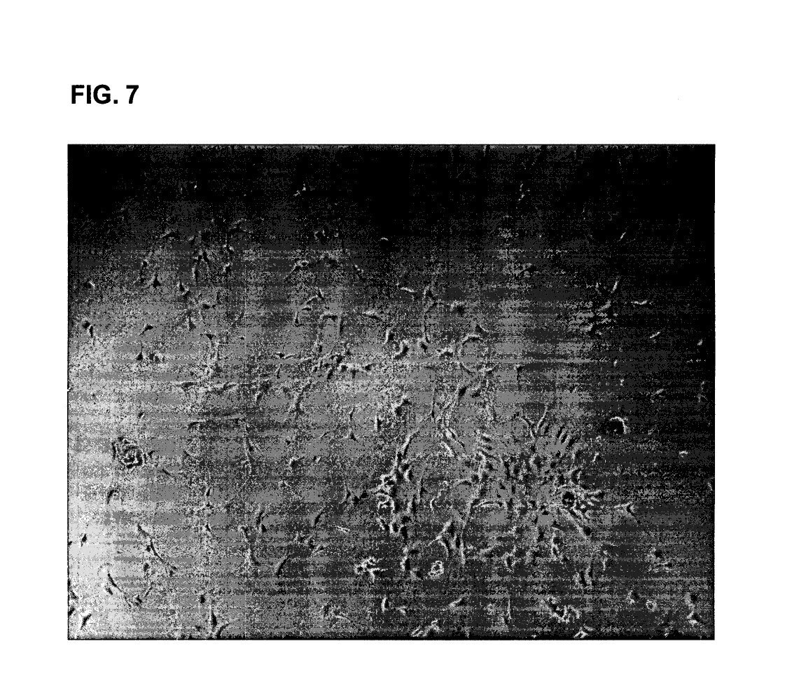

FIG. 7 shows BM-MSCs cultured on the hydrophobic side of a Lumox-biofoil bag. Colony and attached cells observed after 48 h treatment with 10 μM Blebbistatin are shown. A significant number of cells is attached to the hydrophobic surface. Thus, if compared to FIG. 6 (control), treatment with Blebbistatin results in an increased affinity of BM-MSCs to this surface.

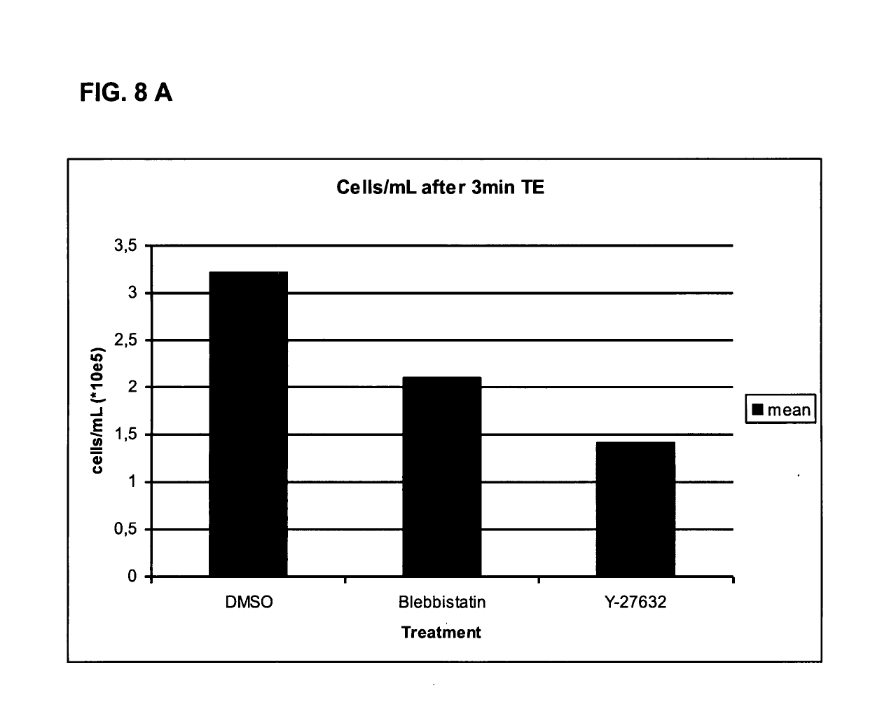

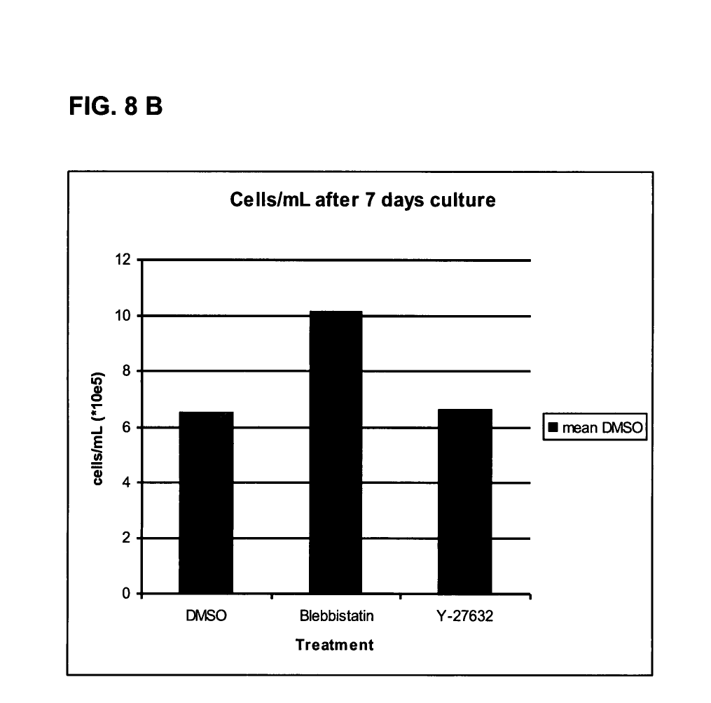

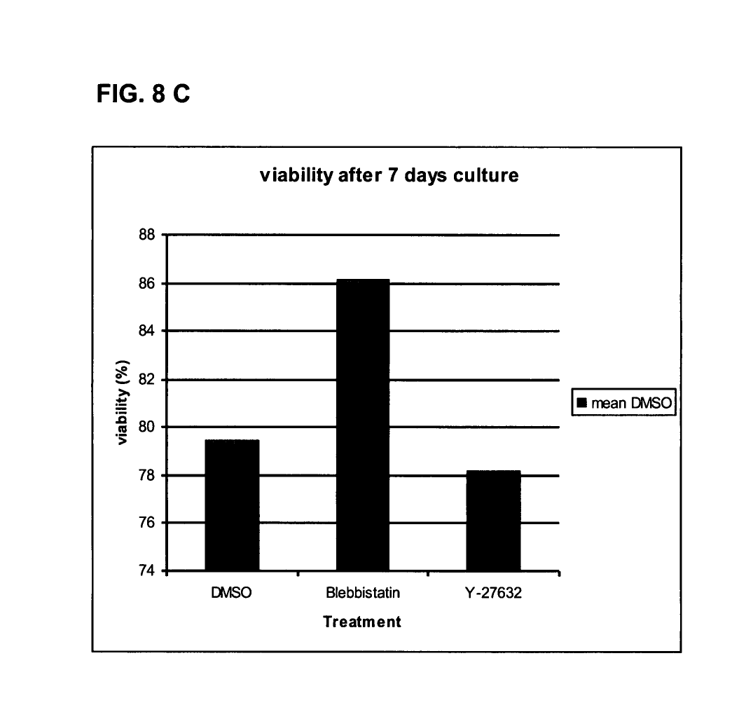

FIG. 8 shows bar diagrams reflecting the effects of Blebbistatin and Y-27632 on mesenchymal stem cells from bone marrow (BM-MSC) while dislocating the cells. Subconfluent BM-MSCs were subjected to 4 h pretreatment (10 μM Blebbistatin or 10 μM Y-27632 vs control) and subsequent incubation for 3 min. with Trypsin/EDTA at 37° C. Cell counts were determined after dislocation of the cells from the surface of tissue culture flasks without specific coating (A). Fewer cells are dislocated under myosin II inhibition than in control condition (without Blebbistatin). Dislocated cells are devoid of aggregates in drug-treated condition. Thus, both Blebbistatin and Y-27632 show protective effects against cell dislocation and clumping, i.e. the cells stay stably attached to the substrate and cannot be easily washed off. Cells were reseeded (all conditions at same density: 5000 cells/cm²) and cultured for additional 7 days in drug-free medium. After 7 days cell counts (B) and viability (C) were determined. If BM-MECs are dislocated from a substrate and reseeded for further culturing, Blebbistatin has a significant protective effect on the cells resulting in enhanced proliferation and viability after 7 days of culturing. An increase in cell proliferation (yield and viability) by Blebbistatin and less for Y-27632 treatment before dislocation in the subsequent drug-free passage (day 7) was also observed.

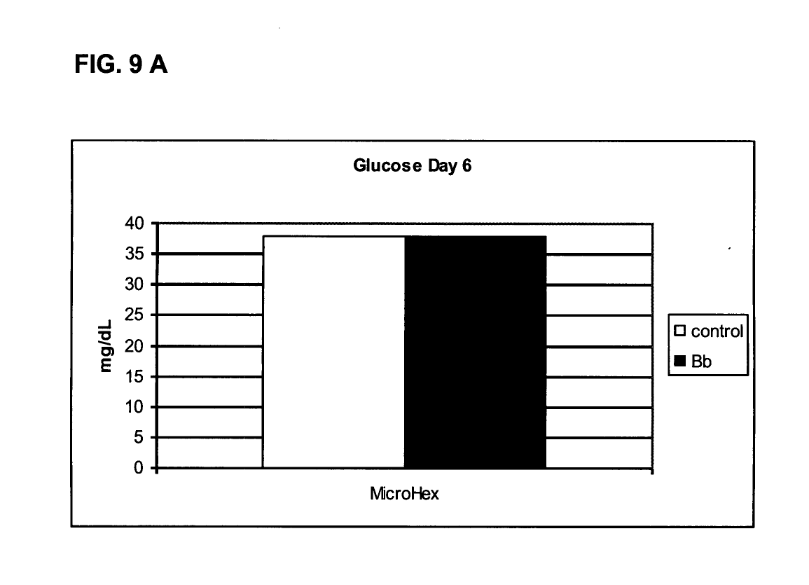

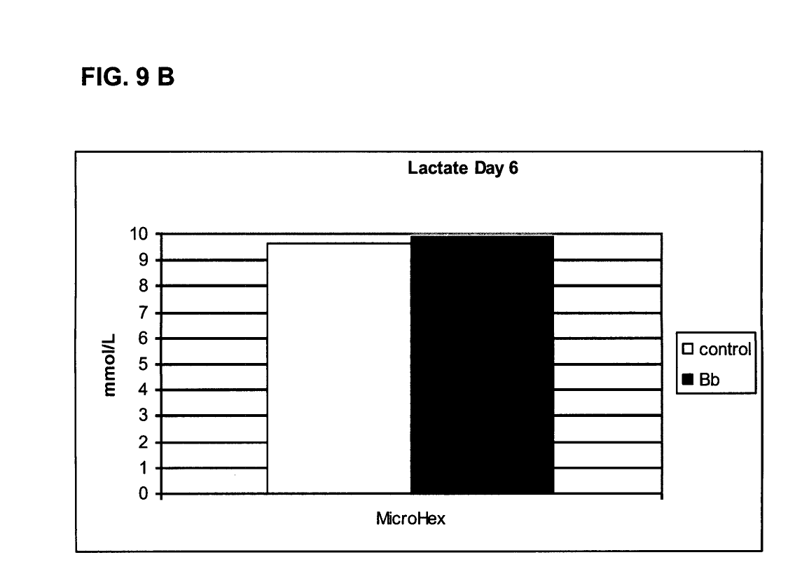

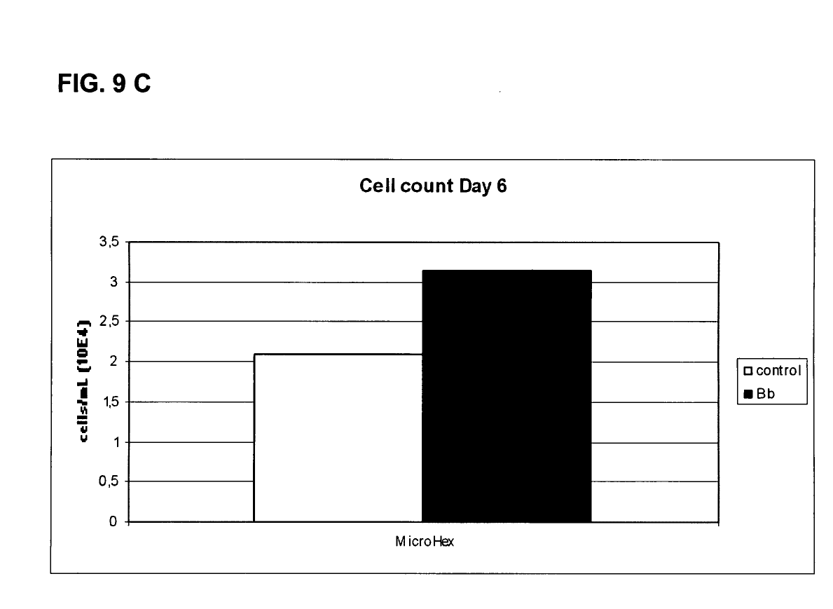

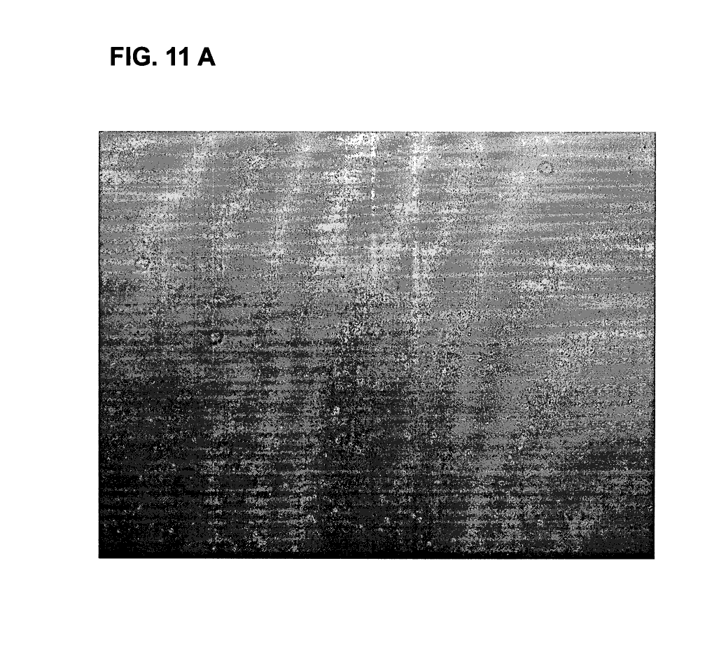

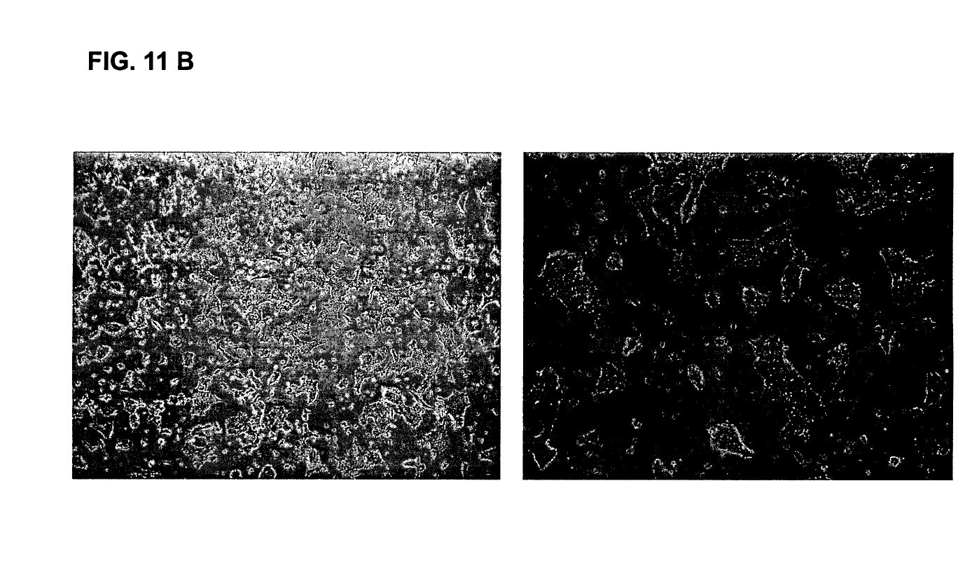

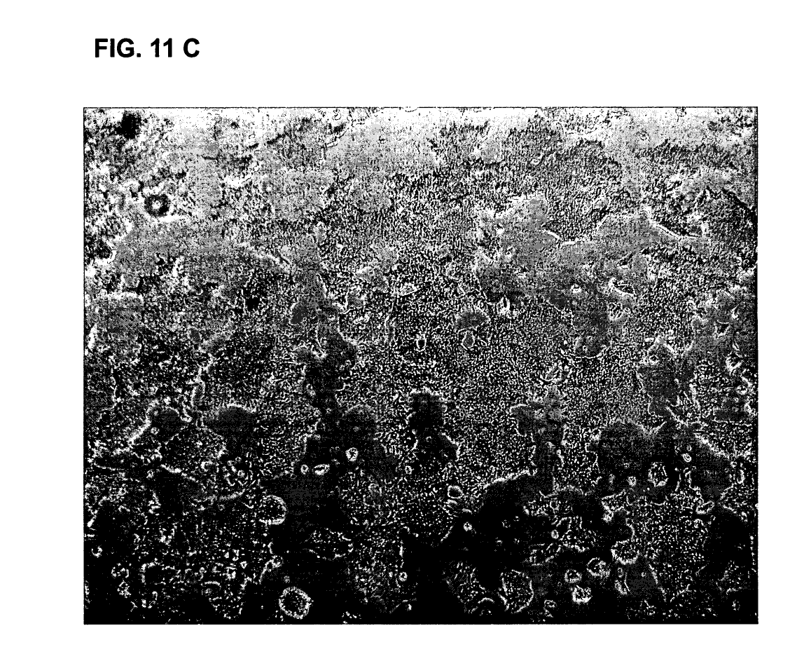

FIG. 9 shows bar diagrams of Glucose consumption (A), Lactate production (B) and cell number (C) of mesenchymal stem cells from bone marrow (BM-MSC) attached to microcarriers. BM-MSCs were seeded on microcarriers for suspension culture in presence or absence of 10 μM Blebbistatin and allowed to proliferate for 6 days. After 24 h incubation without agitation the culture was sampled to determine cell count, Glucose consumption and cell seeding. After 5 additional days of incubation with agitation Glucose consumption (A), Lactate production (B), cell count (C) and cell seeding on carriers (DAPI-stain of cells on carrier, FIG. 11) were determined again. After 24 h cell seeding was markedly increased in presence of Blebbistatin as compared to control. While Blebbistatin seems to have no effect on both Glucose consumption and Lactate production of BM-MSCs after 6 days, cell number is significantly increased after treatment with the inhibitor. Accordingly, Blebbistatin has a protective effect on the cells resulting in enhanced proliferation after 6 days growth on microcarriers.

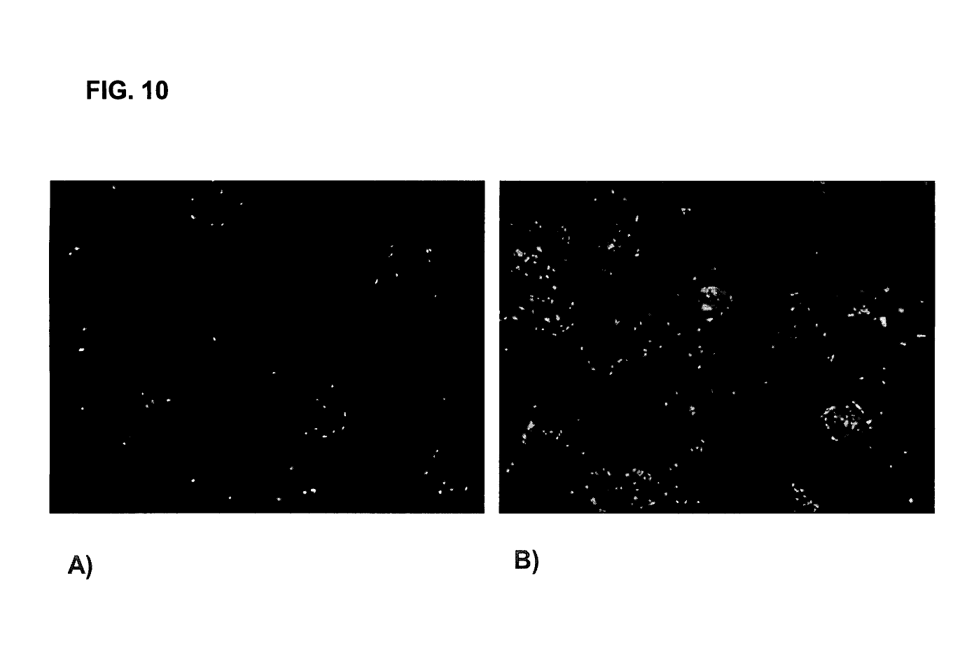

FIG. 10 shows micrographs of the microcarriers according to FIG. 9 after 6 days of incubation (DAPI-stain of BM-MSCs on carriers). Here, it is clearly shown that in presence of Blebbistatin (B) much more cells adhere to the microcarriers than in absence of the inhibitor (A, control).

FIG. 11 shows micrographs of human Embryonic Stem Cells (hESCs) cultured on coated culture dishes. The cells were cultured in defined X-VIVO medium containing no serum or animal-derived components on culture dishes coated with Matrigel®. A: No treatment with inhibitor (control), B: Transient treatment with 10 μM Blebbistatin (left micrograph: 4-fold magnification, right micrograph: 10-fold magnification), C: Culture 1 day after withdrawal of Blebbistatin. The post-split attachment of hESCs in X-VIVO has been low (A: 5-15%). When the culture was split (EDTA passage), the medium was supplemented with 10 μM of Blebbistatin. This increased the attachment of hESCs dramatically (B: 80-95%) compared to the post-split attachment of Blebbistatin-free hESC culture in X-VIVO. In fact, even the individualized hESCs attached very well. The media of the culture was replaced after 24 h with Blebbistatin-free X-VIVO to see how the cells stay attached and proliferate without Blebbistatin. Here, no significant changes could be observed (C).

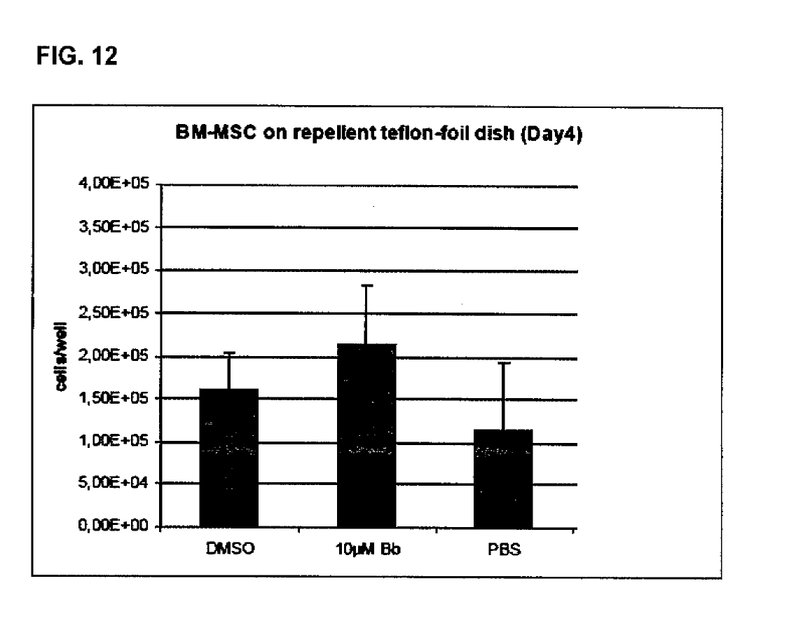

FIG. 12 shows a bar diagram reflecting the effects of Blebbistatin (Bb, (−) enantiomer) on mesenchymal stem cells from bone marrow (BM-MSC). MSCs were cultured with 10 μM Bb for 4 days on repellent Teflon® foil and the cells attached to the foil were counted on day 4. Bars represent the number of cells per well. Control cultures: BM-MSCs in DMSO or PBS without inhibitor. A significant increase in cell yield under myosin inhibition compared to controls was observed. Thus, BM-MSCs attach and proliferate on Teflon® foil.

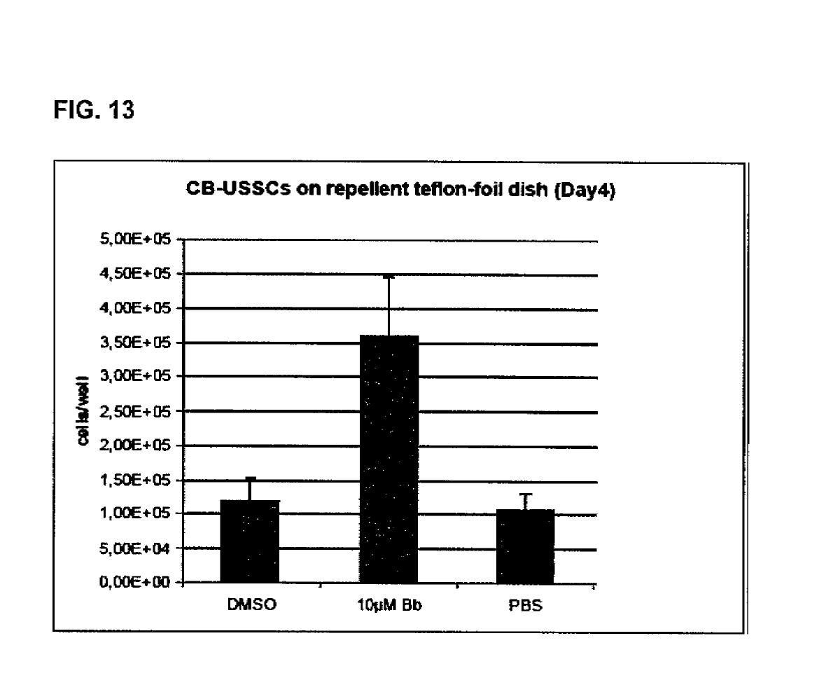

FIG. 13 shows a bar diagram reflecting the effects of Blebbistatin (Bb, (−) enantiomer) on stem cells from cord blood (CB-USSC). USSCs were cultured with 10 μM Bb for 4 days on repellent Teflon® foil and the cells attached to the foil were counted on day 4. Bars represent the number of cells per well. Control cultures: CB-USSCs in DMSO or PBS without inhibitor. A highly significant increase in cell yield under myosin inhibition compared to controls was observed. Thus, CB-USSCs attach and proliferate on Teflon® foil.

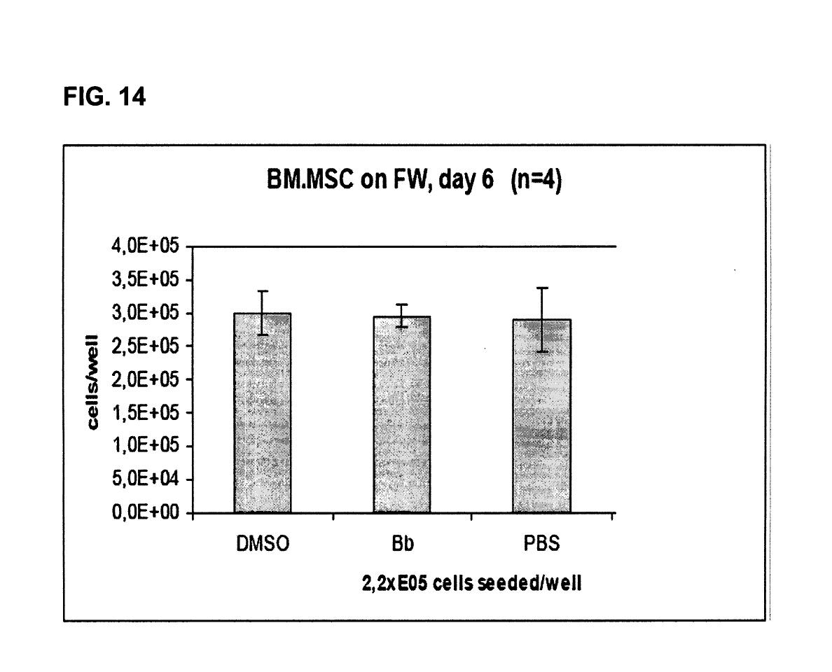

FIG. 14 shows a bar diagram reflecting the effects of Blebbistatin (Bb, (−) enantiomer) on mesenchymal stem cells from bone marrow (BM-MSC). MSCs were cultured with 10 μM Bb on repellent Teflon® foil until confluency was reached, the cells attached to the foil were counted on day 6 after cell seeding. Bars represent the number of cells per well. Control cultures: BM-MSCs in DMSO or PBS without inhibitor. BM-MSCs do not form a completely confluent layer on Teflon® foil. No differences in cell yield under myosin inhibition compared to control, apparent inconsistencies to 4-day experiments might be caused by longer culture period (6 days vs 4 days) and/or the increased initial cell numbers (double amount of cells attached might have been more efficient in secreting ECM to seed onto).

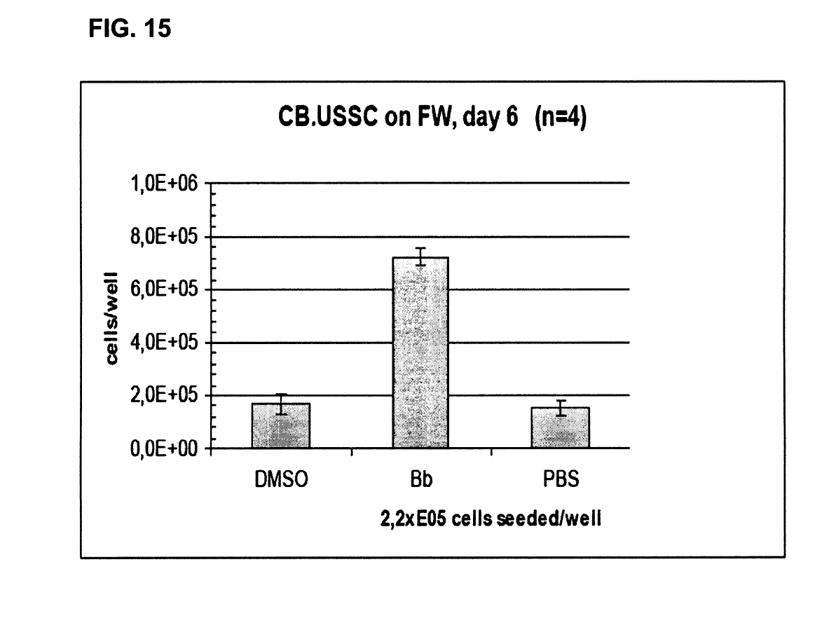

FIG. 15 shows a bar diagram reflecting the effects of Blebbistatin (Bb, (−) enantiomer) on stem cells from cord blood (CB-USSC). USSCs were cultured with 10 μM Bb on repellent Teflon® foil until confluency was reached, the cells attached to the foil were counted on day 6 after cell seeding. Bars represent the number of cells per well. Control cultures: CB-USSCs in DMSO or PBS without inhibitor. USSCs form confluent layers on Teflon foil under myosin inhibition, significant increase in cell yield under myosin inhibition compared to control cultures was observed.

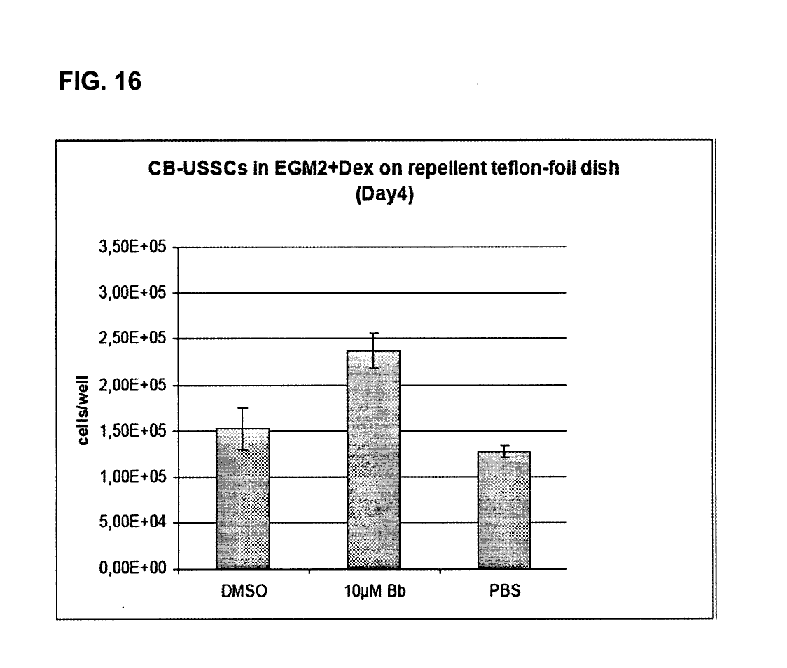

FIG. 16 shows shows a bar diagram reflecting the effects of Blebbistatin (Bb, (−) enantiomer) on stem cells from cord blood (CB-USSC) cultured in low serum EGM2+Dex-media (3% serum). USSCs were cultured with 10 μM Bb on repellent Teflon® foil dish and counted on day 4 after cell seeding. Bars represent the number of cells per well. Control cultures: CB-USSCs in DMSO or PBS without inhibitor. USSCs show significantly enhanced proliferation under myosin-inhibitor treatment. USSC attachment and proliferation on Teflon® foil in serum-reduced (EGM2) media is enhanced by myosin inhibition.

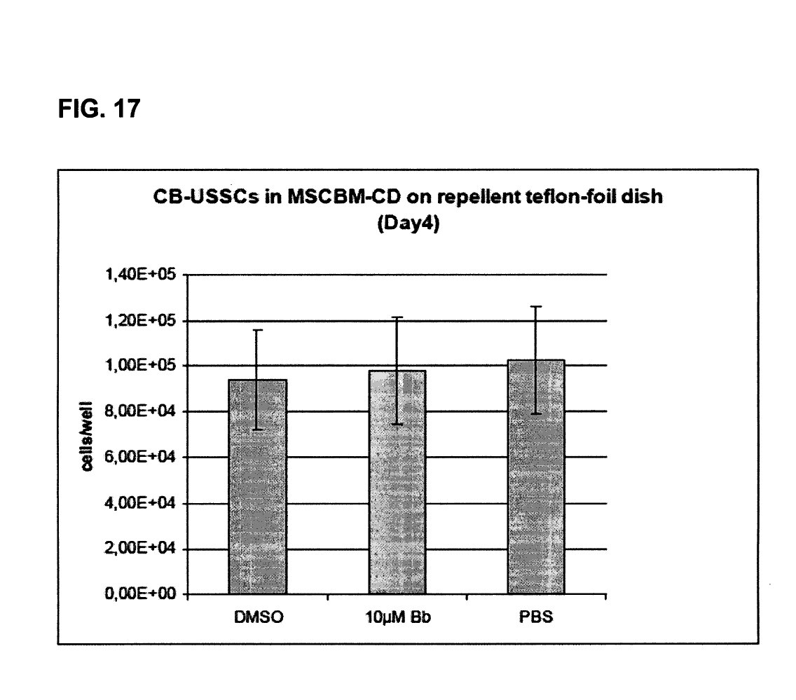

FIG. 17 shows a bar diagram reflecting the effects of Blebbistatin (Bb, (−) enantiomer) on stem cells from cord blood (CB-USSC) cultured in serum-free MSCGM-CD medium. USSCs were cultured with 10 μM Bb on repellent Teflon® foil dish and counted on day 4 after cell seeding. Bars represent the number of cells per well. Control cultures: CB-USSCs in DMSO or PBS without inhibitor. USSC attach but fail to significantly proliferate on Teflon® foil in serum-free medium irrespective of myosin inhibition. No apparent increase in cell yield under myosin inhibition compared to control could be observed.

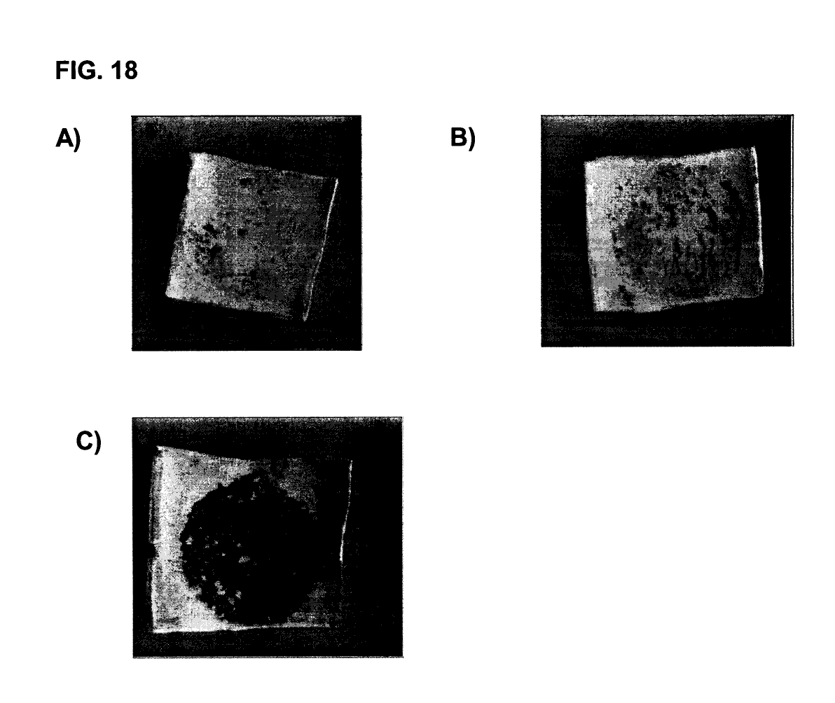

FIG. 18 shows photographs of stained stem cells from cord blood (CB-USSCs) on PTFE vessels (PTFE=polytetrafluoroethylene, trade name (DuPont): Teflon®).
A) DMSO control,
B) PBS control,
C) 10 μM (−) Blebbistatin.

USSCs were cultured with 10 μM Bb for 3 days on repellent PTFE-vessel material compared to control cultures. Markedly enhanced cell spreading but no aggregate formation compared to control was observed under myosin-inhibition.

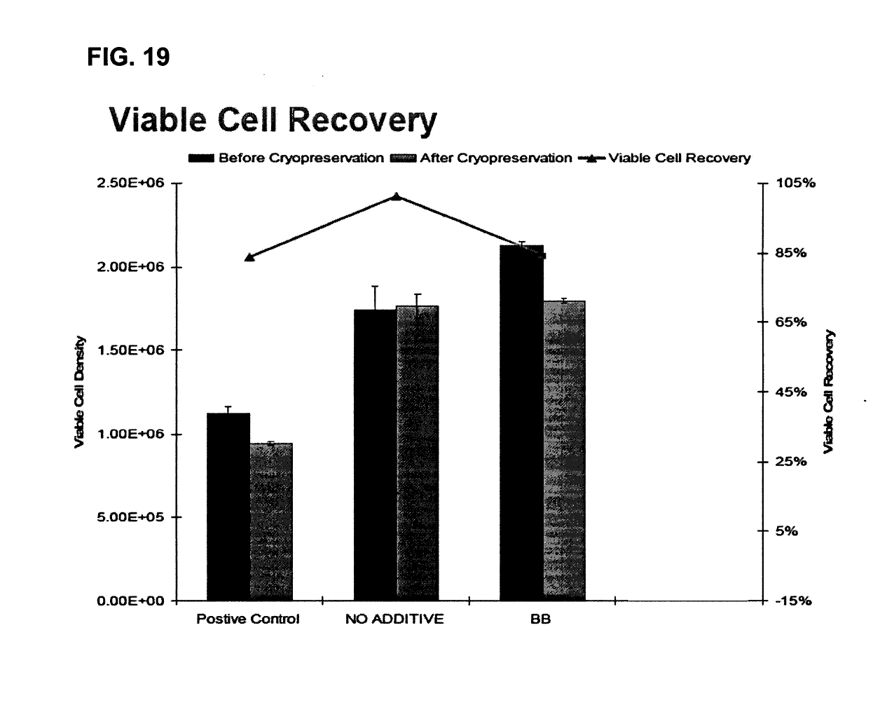

FIG. 19 shows diagrams of viable cell density (bars) and recovery (triangles) of human embryonal stem cells (hEST) before and after cryopreservation with or without myosin-inhibition (BB=Blebbistatin).

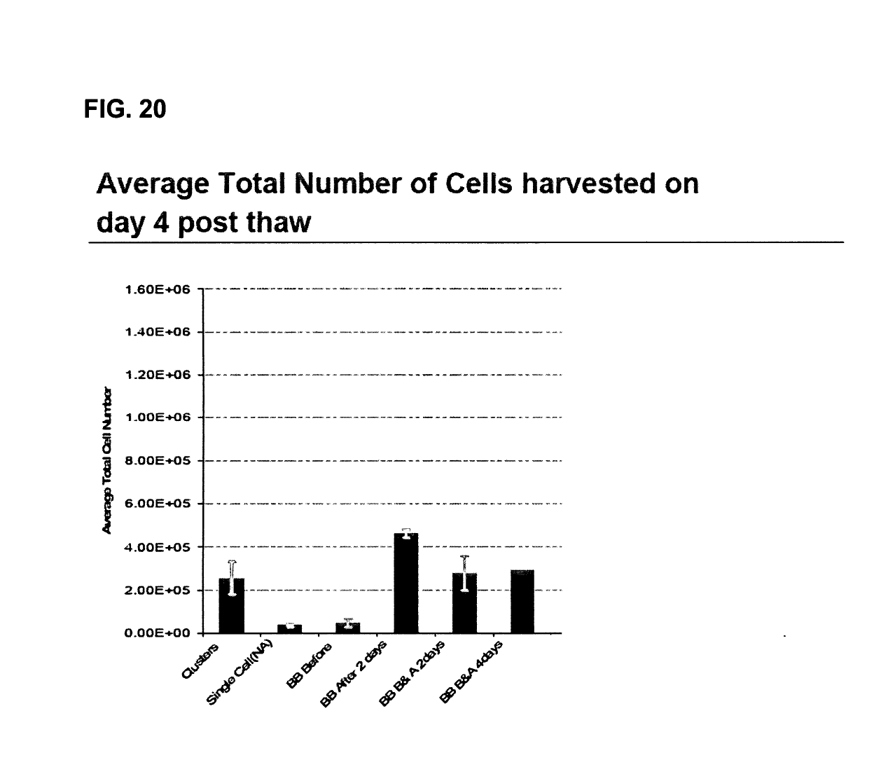

FIG. 20 shows a bar diagram of the average total number of human embryonal stem cells (hEST) 2 and 4 days after thawing cryopreserved cells. Usually only cell clusters can be cryopreserved, but not single ES cells. This figure shows that myosin inhibition significantly enhances single cell survival after cryopreservation.

In summary, FIGS. 19 and 20 show that Blebbistatin increases the cryopreservation recovery of single hESCs to the comparable level of the recovery of hESC clusters. Moreover, adding Blebbistatin before freezing is beneficial for cryopreserving single hESCs.

According to the experimental data shown above, it becomes apparent that direct inhibitors of non-muscle myosin II, such as Blebbistatin, have several positive effects on cell cultures, in particular cultures of attachment-dependent cells. For example, direct myosin II inhibitors
enhance cell survival in suspension culture,
decrease cell aggregation in suspension culture,
promote attachment and proliferation on substrates that are otherwise not suitable for cell culture,
provide a tool for controlling adhesion of cells to a specific substrate,
have a protective effect during cell dislocation,
have a protective effect on cells while freezing and/or thawing them,
have a protective effect during cell sorting (e.g. FACS),
improve initial cell attachment from cord blood and bone marrow MNC-isolates (primary tissue),
myosin inhibition leads to moderate enhanced seeding of BM-MSCs and strong enhanced attachment of CB-USSCs on Teflon® foil,
myosin-inhibition leads to confluent growth on Teflon® with CB-USSCs,
seeding under serum-free conditions in MSC-GM (serum-free medium) is positively affected by myosin inhibition, and
seeding and proliferation under serum-reduced conditions in EGM2-MV is positively affected by myosin inhibition.

It is another aspect of the invention to provide a method for controlling the adhesion of cells to a substrate, in particular attachment-dependent cells, which allows for selecting a suitable substrate for high yield production and low damage detachment of cells. In particular, a method is provided wherein the adhesion of cells to a substrate is controlled by supplying the cells with an inhibitor molecule that directly inhibits non-muscle myosin II. Supplying the cells with the inhibitor enhances the capability of these cells to attach to surfaces to which they otherwise would not have sufficient affinity. Thus, the inhibitor molecules that directly inhibit non-muscle myosin II can be used in an advantageous manner as a kind of trigger which enables the user to control attachment of cells to specific substrates, such as microcarriers for suspension culture. Moreover, the inhibitors according to the invention are useful tools to broaden the choice of suitable substrates for cell cultures so that cell culture methods relying on attachment of cells to microcarriers can be optimized and thus significantly improved.

Preferably, the adhesion of the cells to the substrate is promoted by supplying the cells with the inhibitor molecule. Treating the cells with the inhibitor results in the capability of the cells to attach and proliferate on surfaces that would otherwise not be suitable for culturing these cells. That is, in the method according to the invention the choice of substrates, e.g. microcarriers, suitable for culturing and/or expanding the cells is significantly broadened. Thus, improving and optimizing of culture conditions is facilitated since there are much more options in respect of the substrate to which the cells to be cultured can be adhered.

According to a preferred embodiment of the invention, the substrate is a plurality of microcarriers and the cells attached to said substrate are cultivated under suspension conditions. In this embodiment, the surface area on which the cells can be grown is increased so that the cells can be produced in large quantities on microcarriers, preferably suitable beads, while culturing the cells under suspension conditions, preferably in a suitable bioreactor.

In a preferred embodiment of the invention, the cells are attachment-dependent cells, in particular adult stem cells, preferably mesenchymal stem cells. However, the method according to the invention comprises all cell types whose affinity to a substrate can be influenced by an inhibitor that directly inhibits non-muscle myosin II.

One important aspect of the invention is the use of Blebbistatin for controlling the adhesion of cells to a surface as described above.

In a further aspect of the invention a method for expanding cells is provided, which comprises (a) suspending the cells, (b) allowing the cells to attach to the surface of a plurality of microcarriers, and (c) cultivating the cells attached to the microcarriers under suspension conditions. According to the invention, before and/or while suspending the cells in step (a), the cells are treated with an inhibitor molecule that directly inhibits non-muscle myosin II. In this method, the surface area on which the cells can be grown is significantly increased so that the cells can be produced in large quantities on microcarriers, preferably suitable beads, while culturing the cells under suspension conditions, preferably in a suitable bioreactor. The inhibitor promotes the adhesion of the cells to the microcarriers and enhances the capability of cells, preferably attachment-dependent cells, to attach to surfaces to which they otherwise would have no or not sufficient affinity.

In some aspects, the cells are frozen and thawed before at least one of steps (a) and (b). Surprisingly, freezing and thawing the cells in the course of the method according to the invention results in improvement of adhesion and survival of the cells during expansion so that the yield of cells can be significantly increased. Also here, myosin II inhibitors have a protective effect on the cells when they are frozen and thawed.

According to a preferred embodiment, the cells are attachment-dependent cells, in particular adult stem cells, preferably mesenchymal stem cells. However, the method according to the invention comprises all cell types which can be efficiently expanded in the presence of an inhibitor that directly inhibits non-muscle myosin II.

Another important aspect of the invention is the use of Blebbistatin for expanding cells adhered to a plurality of microcarriers, wherein the cells attached to said microcarriers are cultivated under suspension conditions.

In another aspect of the invention a method for isolating cells from a tissue is provided, comprising (a) dissociating said tissue in the presence of an inhibitor molecule that directly inhibits non-muscle myosin II, and (b) isolating cells of interest from the cells dissociated from the tissue. Surprisingly, it emerged that the inhibitor according to the invention significantly improves survival and recovery of cells from primary isolates. It is therefore an advantage of the method according to the invention that transition of cells from a tissue-embedded to a suspension state is improved. Thus, treating the cells with the inhibitor molecule while isolating them results in higher yields and, occasionally, allows for identification of new cell types out of tissue.

After the cells of interest have been isolated, these cells may be expanded under suspension culture conditions so as to produce a higher number of cells for further use. Preferably, the cells of interest are expanded in the presence of an inhibitor molecule that directly inhibits non-muscle myosin II.

The cells of interest may be attachment-dependent cells, in particular adult stem cells, preferably mesenchymal stem cells. If attachment-dependent cells have been isolated, it may be suitable to expand them under attachment-dependent cell culture conditions in the absence of said inhibitor molecule.

The inhibitor molecule may be Blebbistatin. Blebbistatin is a small molecule which, when applied to cell cultures, minimizes cell death induced by lack of attachment and promotes single cell cultures. In contrast to ROCK inhibitors, Blebbistatin acts as a direct, non-competitive inhibitor of non-muscle myosin II. It does not interfere with a complex signaling cascade but rather directly targets non-muscle myosin II and its interaction with actin by binding to the myosin-ADP-Pi complex. Importantly, Blebbistatin is an easily adjustable and reversible (benign) inhibitor of myosin II. Blebbistatin greatly enhances suspension survival of cells otherwise strictly dependent on attachment. The substance can be added readily to media when required and its effect is completely reversible directly and immediately by removing the substance. The molecule is a crucial tool in the transition towards suspension culture—by minimizing cell death during the initial phase of suspension—as well as in identifying new cells out of tissues—by minimizing cell death during the extraction, i.e. transition from tissue-embedded state or niche to a suspension state.

It is an important aspect of the invention that Blebbistatin can be beneficially used for isolating attachment-dependent cells, in particular to enhance the yield of attachment-dependent cells during the transition from a tissue-embedded state to a suspension state.

Another important aspect of the invention is the use of Blebbistatin to enhance the effect of growth factors on attachment-dependent cells, preferably adult stem cells, in particular mesenchymal stem cells.

A further important aspect of the invention is the use of Blebbistatin for replacing attachment factors during serum-free cultivation of attachment-dependent cells, preferably adult stem cells, in particular mesenchymal stem cells.

Treatment of cells with an inhibitor molecule that directly inhibits non-muscle myosin II, preferably Blebbistatin, essentially influences the characteristics of the cells so that treated cells exhibit new properties and capabilities that are different from those of untreated cells. Consequently, cells treated with myosin II inhibitors are, at least to some extent, a new kind of cells having different morphology and biochemical composition.

Therefore, the invention also comprises a cell, preferably stem cell, more preferred adult stem cell, in particular mesenchymal stem cell, expanded or isolated according to the method of the invention. That is, the invention relates to all cells that are cultured or isolated in the presence of an inhibitor molecule that directly inhibits non-muscle myosin II, preferably Blebbistatin.

Moreover, independent from the method according to the invention, the invention comprises any cell, preferably stem cell, more preferred adult stem cell, in particular mesenchymal stem cell, treated with or cultured in presence of an inhibitor molecule that directly non-muscle myosin II, preferably Blebbistatin.

The invention further comprises a composition comprising at least one cell, preferably stem cell, more preferred adult stem cell, in particular mesenchymal stem cell, according to the invention. In a preferred embodiment of the invention, said composition may comprise a cell culture medium or another solution suitable to ensure viability of the cells. However, the invention is not limited to such compositions. For example, the composition according to the invention may alternatively comprise frozen cells suspended in a suitable protective substance.

The invention also comprises a composition comprising at least one cell, preferably stem cell, more preferred adult stem cell, in particular mesenchymal stem cell, and at least one inhibitor molecule that directly inhibits non-muscle myosin II, preferably Blebbistatin.

In a preferred embodiment of the invention, at least one of the compositions according to the invention is essentially devoid of serum. For example, the composition may comprise a suspension of mesenchymal stem cells in serum-free culture medium including Blebbistatin.

The cells and/or compositions according to the invention may be included in a Kit for non-medical laboratory use or as part of a pharmaceutical preparation.

### LITERATURE

Straight et al.: Science, 2003, 299, 1743-1747
Walker, A., Su, H., Conti, M. A., Harb, N., Adelstein, R. S., Sato, N.: Nature Communications, 2010, DCI:10.1038, ncomms1074

1. Method for promoting adhesion of cells to a substrate comprising supplying the cells with the non-muscle myosin II inhibitor Blebbistatin, wherein the adhesion of the cells to the substrate is promoted and the cells are capable to attach to surfaces to which they otherwise would not have sufficient affinity.

2. The method of claim 1, wherein said substrate is a non-stick material or at least in part coated with a non-stick material.

3. The method of claim 2, wherein said non-stick material comprises polytetrafluoroethylene (PTFE), polyfluoroethylene (PFE), perfluoroalkoxy (PFA), fluorinated ethylene propylene (FEP), a titanium dioxide compound or a titanium dioxide comprising composition, or any combination thereof.

4. The method of claim 1, wherein said substrate is a surface of a medical device.

5. The method of claim 1, wherein said substrate is a plurality of microcarriers.

6. The method of claim 1, wherein said cells are attachment-dependent cells.

7. The method of claim 1, wherein said cells are expanded under suspension culture conditions.

8. The method of claim 1, wherein said cells are suspended in a serum-reduced or serum-free culture medium.

9. The method of claim 1, wherein the cells are frozen and/or thawed in the presence of Blebbistatin and then brought in contact with said substrate.

10. The method of claim 4, wherein the medical device is a stent, a patch or an artificial blood vessel or organ.

11. The method of claim 1, wherein said attachment-dependent cells are adult stem cells.

12. Device comprising at least one surface and the surface is coated with cells that usually have no or only low affinity to this surface.

13. The device of claim 12, wherein said surface comprises a non-stick material.

14. The device of claim 12, wherein said cells are attachment-dependent cells.

15. The device of claim 12, wherein said device is a medical device.

16. The method of claim 11, wherein said adult stem cells are mesenchymal stem cells.

17. The device of claim 13, wherein the non-stick material is polytetrafluoroethylene (PTFE), polyfluoroethylene (PFE), perfluoroalkoxy (PFA), fluorinated ethylene propylene (FEP), a titanium dioxide compound or a titanium dioxide comprising composition, or any combination thereof.

18. The device of claim 14, wherein the attachment-dependent cells are adult stem cells.

19. The device of claim 18, wherein the adult stem cells are mesenchymal stem cells.

20. The device of claim 15, wherein the medical device is a stent, a patch or an artificial blood vessel or organ.
